(19) United States
(12) Patent Application Publication
(10) Pub. No.: US 2015/0094583 A1
(43) Pub. Date: Apr. 2, 2015

(54) ULTRASONIC MEASUREMENT APPARATUS AND ULTRASONIC MEASUREMENT METHOD

(71) Applicant: SEIKO EPSON CORPORATION, Tokyo (JP)

(72) Inventor: Robina ATSUCHI, Fujimi-machi (JP)

(21) Appl. No.: 14/487,766

(22) Filed: Sep. 16, 2014

(30) Foreign Application Priority Data
Sep. 30, 2013 (JP) 2013-203606

(51) Int. Cl. A61B 8/00; A61B 8/08; A61B 8/14; G01N 29/24; G01N 29/22

(52) U.S. Cl. CPC A61B 8/4254; G01N 29/24; G01N 29/226; A61B 8/4444; A61B 8/14; A61B 8/5207; A61B 8/4477; A61B 8/4227; G01N 2291/105; G01N 2291/0289; G01N 2291/018
USPC 600/443; 600/459

(57) ABSTRACT

An ultrasonic measurement apparatus includes: an ultrasonic probe that transmits an ultrasonic wave and outputs a received signal based on a reflected wave of the ultrasonic wave; a belt that fixes the ultrasonic probe to a subject; a position detecting unit that detects position information of the ultrasonic probe; a transmission direction determining unit that determines a transmission direction of the ultrasonic prove based on the position information; a transmission signal generating unit that generates a signal for transmitting the ultrasonic wave in the determined transmission direction; a received signal correcting unit that corrects the received signal of the reflected wave corresponding to the transmitted ultrasonic wave based on the position information; and a signal combining unit that combines the received signal.

### BACKGROUND

1. Technical Field

The present invention relates to an ultrasonic measurement apparatus and an ultrasonic measurement method.

2. Related Art

As an apparatus that transmits an ultrasonic wave toward a target and detects a reflected wave from the interface having different acoustic impedance inside the target, for example, an ultrasonic diagnostic apparatus for medical use for examining the inside of the body is known.

The ultrasonic diagnostic apparatus for medical use emits an ultrasonic wave to the inside of the body of the subject from the outside of the body, detects a reflected ultrasonic wave that is reflected from each part having different acoustic impedance in the body, generates a tomographic image of the inside of the body based on the detection signal, and displays the tomographic image on the display screen.

In a general ultrasonic examination, an examiner, such as a doctor, holds an ultrasonic probe to transmit and receive an ultrasonic wave and performs an ultrasonic scan in a state where an ultrasonic wave transmission and reception portion located at the distal end of the probe is pressed against a desired part of the body surface of the subject. Since an ultrasonic tomographic image on the display screen is updated at predetermined intervals, the examiner checks the tomographic image while changing the position, angle, and the like of the ultrasonic probe, determines the position and the angle of the ultrasonic probe so that a desired tomographic image is obtained, and then continues the observation of the image in this state for a predetermined time.

In a state where the examiner himself or herself holds the ultrasonic probe, a positional shift or angle change of the ultrasonic probe occurs due to hand shaking of the examiner, movement of the subject, and the like. For this reason, JP-A-2011-101679 discloses a system that acquires an ultrasonic tomographic image of a desired part by setting a plurality of ultrasonic probes in a holder, in which probe positions are defined, and bringing the holder into contact with the desired part.

However, in the holder disclosed in JP-A-2011-101679, the shape and the probe positions where ultrasonic probes are set are fixed. Accordingly, since a region whose tomographic image can be obtained using an ultrasonic wave is determined by the shape and the probe position of the holder, the region can not be selected freely.

### SUMMARY

An advantage of some aspects of the invention is to easily acquire a tomographic image of a desired part of a subject using an ultrasonic wave.

The invention can be implemented as the following forms or application examples.

### Application Example 1

This application example is directed to an ultrasonic measurement apparatus including: a plurality of ultrasonic probes that transmit ultrasonic waves based on transmission signals and output received signals based on reflected waves of the ultrasonic waves; a fixture that fixes the plurality of ultrasonic probes to a subject; a position detecting unit that detects position information indicating positions of the plurality of ultrasonic probes; a transmission direction determining unit that determines a transmission direction for each of the ultrasonic probes based on the position information; a transmission signal generating unit that generates for each ultrasonic probe a transmission signal for deflecting the ultrasonic waves in the determined transmission direction; a received signal correcting unit that corrects the received signals based on the position information; and a signal combining unit that combines the received signals corrected for the ultrasonic probes.

According to such a configuration, when a plurality of ultrasonic probes are fixed to the subject using the fixture, the transmission direction of the ultrasonic wave transmitted from each ultrasonic probe changes with the position of the ultrasonic probe in contact with the subject. However, the transmission direction is determined according to the position information of the ultrasonic probe detected by the detecting unit, the transmission signal deflected in the determined transmission direction is generated, and the ultrasonic wave is transmitted from each ultrasonic probe based on the generated transmission signal. Therefore, it is possible to control the directions of ultrasonic waves transmitted from the plurality of ultrasonic probes regardless of the position of each ultrasonic probe. In addition, since the received signals of the reflected waves of the ultrasonic waves are corrected and combined according to the position information, it is possible to easily combine the received signals regardless of the position of each ultrasonic probe. Therefore, since the examiner does not need to continuously pay attention to the shape of the body of the subject and the contact state of the ultrasonic probe or press the plurality of ultrasonic probes against the subject while holding the plurality of ultrasonic probes, it is possible to reduce the burden of the examiner for the ultrasonic examination in addition to increasing the degree of freedom to select the examination point using the ultrasonic wave.

### Application Example 2

In the ultrasonic measurement apparatus according to the application example described above, it is preferable that the transmission signal generating unit generates the transmission signal including performing delay processing.

According to such a configuration, since a transmission signal subjected to delay processing is generated, it is possible to deflect the wavefront of the ultrasonic wave by controlling the phase of the transmission signal.

### Application Example 3

In the ultrasonic measurement apparatus according to the application example described above, it is preferable that the received signal correcting unit performs delay correction processing on the received signal of each of the ultrasonic probes based on the position information.

According to such a configuration, it is possible to match the phases of the received signals to each other by performing delay correction processing on the received signals.

### Application Example 4

In the ultrasonic measurement apparatus according to the application example described above, it is preferable that the position detecting unit detects an installation angle of each of the ultrasonic probes with respect to the subject and calculates the position information based on the detected installation angle.

### Application Example 5

In the ultrasonic measurement apparatus according to the application example described above, it is preferable that the position detecting unit detects a difference between installation angles of the ultrasonic probes adjacent to each other and calculates the position information based on the detected difference.

### Application Example 6

In the ultrasonic measurement apparatus according to the application example described above, it is preferable that the ultrasonic measurement apparatus further includes a display processing unit that generates an image based on the received signal combined by the signal combining unit and displays the generated image.

According to such a configuration, an image is generated by combining the received signals based on the reflected waves of the ultrasonic waves transmitted from the ultrasonic probes, and the generated image is displayed. Therefore, it is possible to display a tomographic image based on the plurality of ultrasonic waves.

### Application Example 7

This application example is directed to an ultrasonic measurement method including: detecting position information of a plurality of ultrasonic probes fixed to a subject by a fixture; determining a transmission direction for each of the ultrasonic probes based on the position information; generating a transmission signal for deflecting each of the ultrasonic waves in the determined transmission direction for each of the ultrasonic probes; transmitting the ultrasonic waves based on the transmission signal; acquiring received signals based on reflected waves of the transmitted ultrasonic waves; correcting the received signals based on the position information; and combining the received signals corrected for the ultrasonic probes.

According to such a method, when a plurality of ultrasonic probes are fixed to the subject by the fixture, the transmission direction of the ultrasonic wave transmitted from each ultrasonic probe changes with the position of the ultrasonic probe in contact with the subject. However, the transmission direction is determined according to the position information of the ultrasonic probe detected by the detecting unit, the transmission signal deflected in the determined transmission direction is generated, and the ultrasonic wave is transmitted from each ultrasonic probe based on the generated transmission signal. Therefore, it is possible to control the directions of ultrasonic waves transmitted from the plurality of ultrasonic probes regardless of the position of each ultrasonic probe. In addition, since the received signals of the reflected waves of the ultrasonic waves are corrected and combined according to the position information, it is possible to easily combine the received signals regardless of the position of each ultrasonic probe. Therefore, since the examiner does not need to continuously pay attention to the shape of the body of the subject and the contact state of the ultrasonic probe or press the plurality of ultrasonic probes against the subject while holding the plurality of ultrasonic probes, it is possible to reduce the burden of the examiner for the ultrasonic examination in addition to increasing the degree of freedom of the examination points using the ultrasonic wave.

### BRIEF DESCRIPTION OF THE DRAWINGS

The invention will be described with reference to the accompanying drawings, wherein like numbers reference like elements.

FIG. 1 is a block diagram showing the functional configuration of an ultrasonic measurement apparatus according to an embodiment of the invention.

FIG. 2A is a diagram showing an application example of a measurement unit, FIG. 2B is a cross-sectional view taken along the line A-A of FIG. 2A, and FIG. 2C is an enlarged view.

FIG. 3 is a diagram showing a difference in traveling direction of the ultrasonic beam between a plurality of ultrasonic probes.

FIG. 4 is a diagram showing the configuration of an ultrasonic element array.

FIG. 5 is a diagram showing a driving circuit of the ultrasonic element array.

FIG. 6 is a flowchart illustrating the flow of the process of the ultrasonic measurement apparatus according to the embodiment of the invention.

### DESCRIPTION OF EXEMPLARY EMBODIMENTS

Hereinafter, an embodiment of the invention will be described with reference to the accompanying diagrams.

### Embodiment

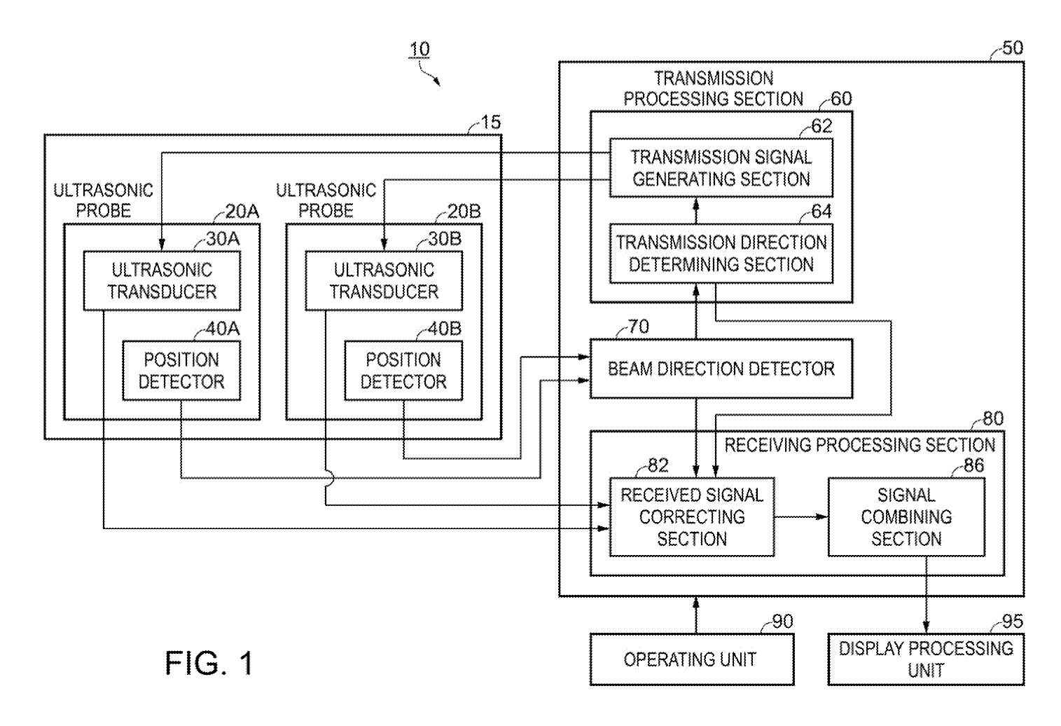

FIG. 1 is a block diagram showing the functional configuration of an ultrasonic measurement apparatus 10 according to the present embodiment. The ultrasonic measurement apparatus 10 includes a measurement unit 15, a measurement control unit 50, an operating unit 90, and a display processing unit 95. The ultrasonic measurement apparatus 10 has a function of performing an ultrasonic scan using the measurement unit 15 mounted on the body of the subject, processing a signal of a reflected wave obtained by the ultrasonic scan, and displaying a tomographic image of the inside of the body of the subject.

### 1. Measurement Unit

The measurement unit 15 includes a plurality of ultrasonic probes 20A and 20B. In FIG. 1, two ultrasonic probes 20A and 20B are representatively shown. The ultrasonic probes 20A and 20B include ultrasonic transducers 30A and 30B, respectively. Each of the ultrasonic transducers 30A and 30B has a function of transmitting an ultrasonic wave based on an electrical signal (transmission signal), detecting a reflected wave of the ultrasonic wave, and outputting the reflected wave as an electrical signal (received signal).

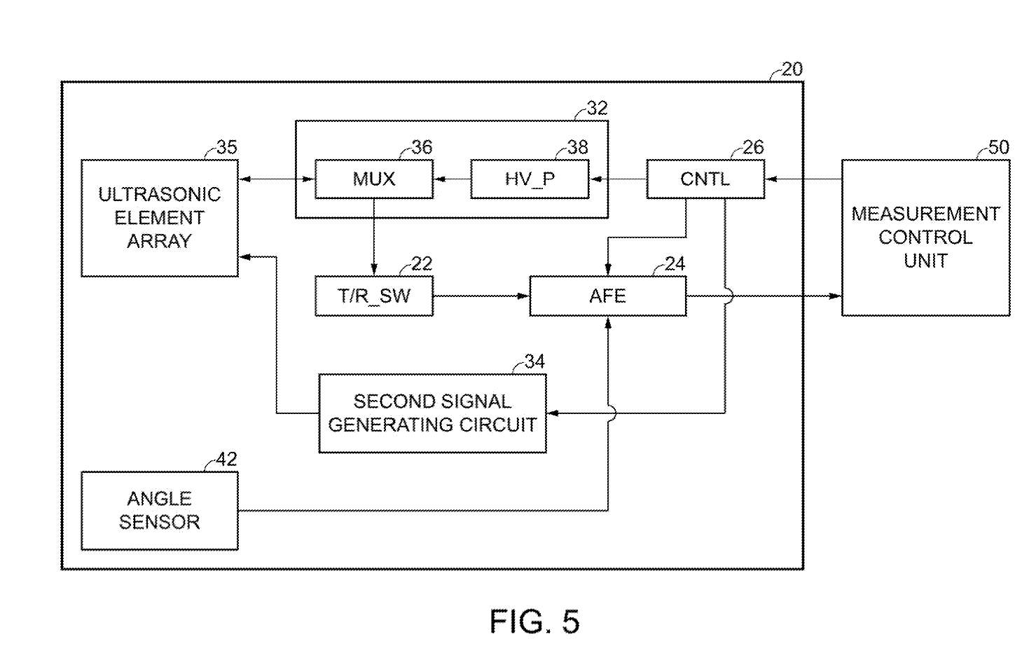

The ultrasonic probes 20A and 20B include position detectors 40A and 40B, respectively. The position detectors 40A and 40B detect position information indicating the positions of the ultrasonic probes 20A and 20B, respectively. In the present embodiment, the position detectors 40A and 40B detect the installation angles of the ultrasonic probes 20A and 20B corresponding to the positions, respectively, as position information, when an angle sensor 42 (FIG. 5), such as an acceleration sensor, an angular velocity sensor, or a gyro sensor, is used. As the installation angle, an angle relative to the direction of gravity is assumed. However, the installation angle is not limited to this.

In the present embodiment, it is assumed that the ultrasonic probes 20A and 20B include the position detectors 40A and 40B, respectively. However, the invention is not limited to this. For example, in the ultrasonic probes 20A and 20B, a detection unit that detects a difference in installation angle between one ultrasonic probe 20A and the other ultrasonic probe 20B as position information can also be assumed.

In addition, the position information detected by each of the position detectors 40A and 40B is transmitted to the measurement control unit 50.

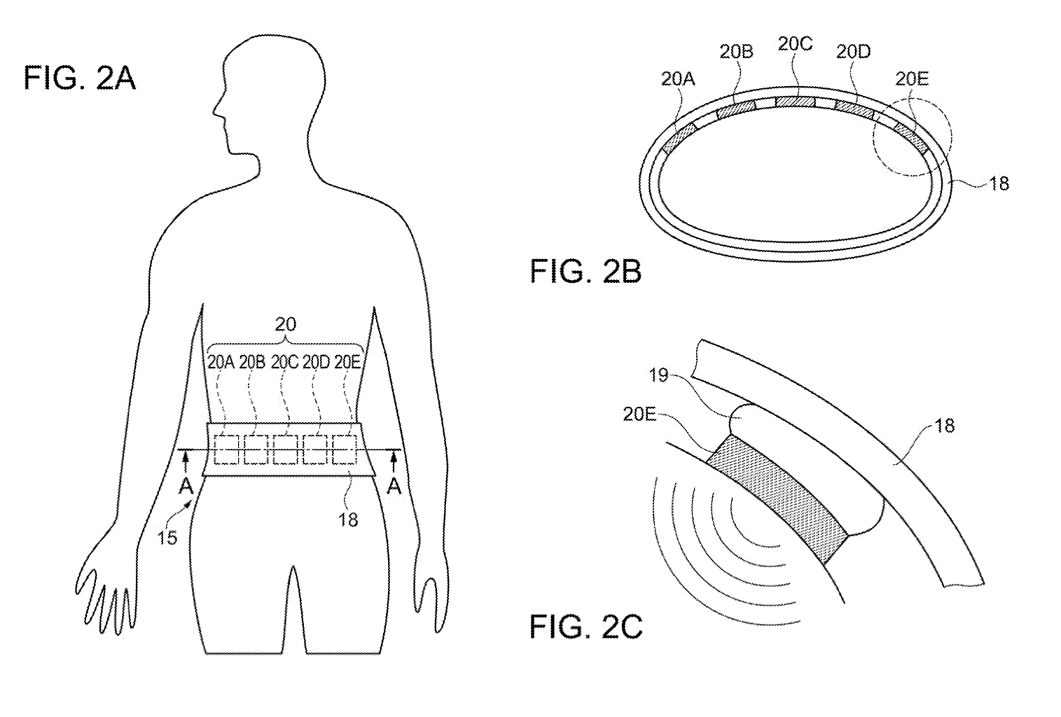

As shown as an application example in FIG. 2A, the measurement unit 15 includes a belt 18 that can be mounted on the body of the subject. The belt 18 is a fixture for fixing the ultrasonic probes 20A and 20B to the body of the subject. As shown in FIG. 2B that is a cross-sectional view taken along the line A-A of FIG. 2A, a plurality of ultrasonic probes 20 are provided at predetermined intervals on a side facing the body of the subject.

In the present embodiment, as shown in FIG. 2C, for example, a bag-shaped rubber member 19 filled with air or the like is interposed between the belt 18 and each ultrasonic probe 20, and each ultrasonic probe 20 is fitted and fixed to the body of the subject. Each ultrasonic probe 20 is configured so as to transmit a beam of the ultrasonic wave in a reference beam direction from the surface in contact with the body, that is, in a direction that is approximately perpendicular to the probe surface of the ultrasonic probe 20 and is toward the inside of the body of the subject.

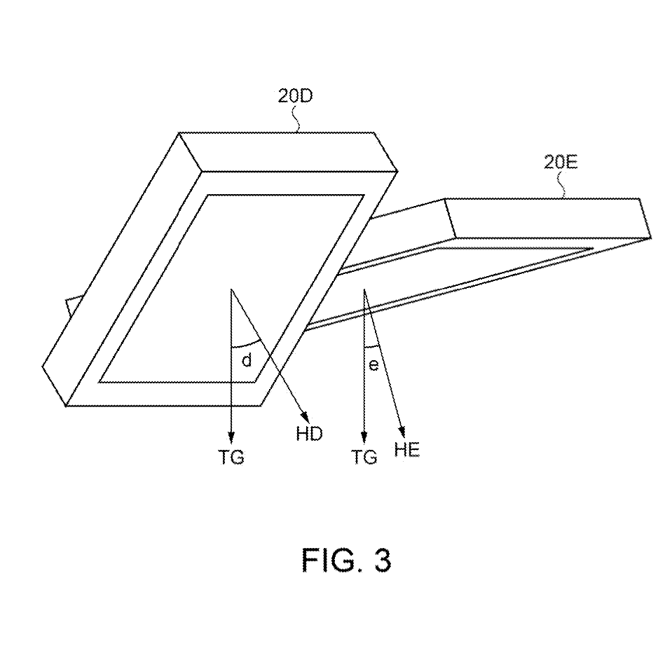

When the belt 18 is mounted on the body, as shown in FIG. 3, cases are assumed in which each of two ultrasonic probes 20D and 20E is in contact with the body in a twisted state or a gap is generated between each of two ultrasonic probes 20D and 20E and the body. As a result, a difference occurs in the traveling direction of the beam of the ultrasonic wave in the two ultrasonic probes 20D and 20E. For example, assuming that the traveling target of the ultrasonic wave transmitted from each ultrasonic probe 20 is a target direction (transmission direction) TG, a difference of an angle d with respect to a reference beam direction HD occurs in the ultrasonic probe 20D. In addition, a difference of an angle e with respect to a reference beam direction HE occurs in the ultrasonic probe 20E.

In the present embodiment, when a transmission signal indicating the correction of the beam direction is received from the measurement control unit 50, the ultrasonic probe 20 can focus the beam in a direction different from the transmission angle of each reference beam direction by performing delay processing on the ultrasonic wave based on the transmission signal.

For example, when a transmission signal indicating the correction of the angle d for the beam direction is received from the measurement control unit 50, the ultrasonic probe 20D controls the phase of the ultrasonic wave by performing delay processing and transmits an ultrasonic wave deflected in the target direction TG that is different from the reference beam direction HD by the angle d. Similarly, when a transmission signal indicating the correction of the angle e for the beam direction is received from the measurement control unit 50, the ultrasonic probe 20E controls the phase of the ultrasonic wave by performing delay processing and transmits an ultrasonic wave deflected in the target direction TG that is different from the reference beam direction HE by the angle e.

The reference beam direction of the ultrasonic probe 20 is defined by position information detected by each position detector 40 (not shown). Similarly, also in the case of three or more ultrasonic probes 20, the reference beam direction can be acquired based on each piece of the position information.

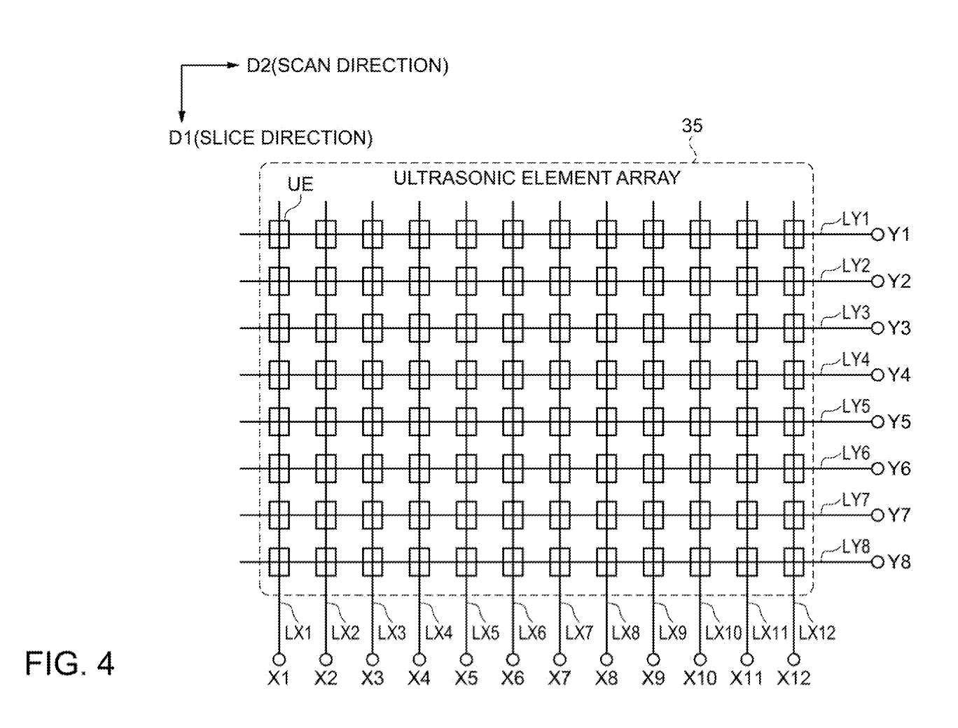

Here, a method of driving the ultrasonic transducers 30A and 30B will be described with reference to FIG. 4 showing the configuration of an ultrasonic element array 35 and FIG. 5 showing a driving circuit of the ultrasonic element array.

Each of the ultrasonic transducers 30A and 30B includes the ultrasonic element array 35 in which ultrasonic elements UE, such as piezoelectric elements, are arrayed in a matrix and wiring lines are provided for columns and rows to scan beams in a column direction and a row direction.

The ultrasonic probe 20 includes a first signal generating circuit 32, a second signal generating circuit 34, a transmission and reception changeover switch (T/R_SW) 22, an analog front end (AFE) 24, and a control circuit (CNTL) 26, as circuits to drive the ultrasonic element array 35.

Each of the first signal generating circuit 32 and the second signal generating circuit 34 includes a multiplexer (MUX) 36 or a pulse signal generator (HV_P) 38. The MUX 36 performs channel switching between the received signal and the driving voltage for driving the ultrasonic transducers 30A and 30B. The HV_P 38 generates a signal (pulse) for driving the ultrasonic element UE.

The T/R_SW 22 performs switching between a signal at the time of transmission and a signal at the time of reception. The AFE 24 has functions of amplifying a received signal, gain setting, frequency setting, and A/D conversion. The CNTL 26 performs control of the phase or frequency of the driving signal for the HV_P 38, voltage gradient control of the driving voltage for the second signal generating circuit 34, and angle calculation processing or position information transmission processing based on the output signal from the angle sensor 42.

The first signal generating circuit 32 supplies first driving voltages VDR1 to VDR12 to first to twelfth first direction terminals X1 to X12. The second signal generating circuit 34 supplies second driving voltages VCOM1 to VCOM8 having different voltages to the first to eighth second direction terminals Y1 to Y8.

The first signal generating circuit 32 and the second signal generating circuit 34 can generate a beam of the ultrasonic wave and control the transmission direction of the generated beam by appropriately controlling the first driving voltages VDR1 to VDR12 and the second driving voltages VCOM1 to VCOM8 based on the transmission signal transmitted from the measurement control unit 50.

For example, the first signal generating circuit 32 and the second signal generating circuit 34 deflect the beam of the ultrasonic wave in a desired direction, focus the beam electronically, or scan the direction of the beam in a scan direction D2 by setting a time difference for the timing, at which the first driving voltages VDR1 to VDR12 and the second driving voltages VCOM1 to VCOM8 are supplied, to delay the timing or by setting a voltage gradient in the second driving voltages VCOM1 to VCOM8. The details of the control method of the first signal generating circuit 32 and the second signal generating circuit 34 are disclosed in JP-A-2006-61252, for example.

### 2. Measurement Control Unit

Referring back to FIG. 1, the measurement control unit 50 will be described. The measurement control unit 50 includes a transmission processing section 60, a beam direction detector 70, and a receiving processing section 80. In the present embodiment, the measurement control unit 50 includes a CPU, a RAM, a ROM, a storage device, and the like (not shown) as hardware. The function of each functional unit is realized by making these hardware components and software stored in the ROM or the storage device cooperate with each other.

The beam direction detector 70 acquires the position information transmitted from the position detectors 40A and 40B, and detects the direction of the reference beam transmitted from the ultrasonic probes 20A and 20B based on the acquired position information. The information regarding the direction of the reference beam detected by the beam direction detector 70 is transmitted to the transmission processing section 60 and the receiving processing section 80.

The transmission processing section 60 includes a transmission signal generating section 62 and a transmission direction determining section 64.

The transmission direction determining section 64 determines a target direction, in which the ultrasonic wave is transmitted, for each of the ultrasonic probes 20A and 20B based on the measurement target and the information regarding the direction of the reference beam of the ultrasonic probes 20A and 20B. The measurement target is a target portion inside the body of the subject that is determined in advance by operating the operating unit 90 by the examiner. The transmission direction determining section 64 determines a target direction for deflecting the beam of the ultrasonic wave, which is transmitted from the ultrasonic probes 20A and 20B, to the measurement target, and transmits target direction information indicating the target direction to the transmission signal generating section 62 and the receiving processing section 80.

The transmission signal generating section 62 generates a transmission signal, which is to be transmitted to each of the ultrasonic probes 20A and 20B, based on the target direction information, and transmits the generated transmission signal to each of the ultrasonic probes 20A and 20B. The ultrasonic transducers 30A and 30B transmit the ultrasonic waves so as to be deflected in the target direction based on the respective transmission signals. As a result, each beam of the transmitted ultrasonic wave is deflected in the target direction.

In addition, the transmission signal may indicate the connection of the line focus or the point focus to the measurement target by controlling the delay of the driving voltage supplied to the ultrasonic transducers 30A and 30B as well as indicating the target direction of the ultrasonic wave transmitted.

The receiving processing section 80 includes a received signal correcting section 82 and a signal combining section 86.

The received signal correcting section 82 corrects a received signal based on a reflected wave when the ultrasonic wave transmitted from each of the ultrasonic probes 20A and 20B is reflected at the interface of different acoustic impedances in apart within the body. In the present embodiment, the received signal correcting section 82 matches the phases of received signals by performing delay correction processing for correcting the time delay on the received signal for the ultrasonic probes 20A and 20B based on the information regarding the reference beam direction or the target direction information. The signal combining section 86 combines (phase addition) the received signals of the ultrasonic probes 20A and 20B after the delay correction processing. The receiving processing section 80 transmits the received signal to the display processing unit 95 after performing filtering processing, amplification processing, detection processing, and the like on the combined received signal.

The display processing unit 95 generates an image signal, such as a tomographic image, based on the received signal and displays the generated image signal on a display device or the like.

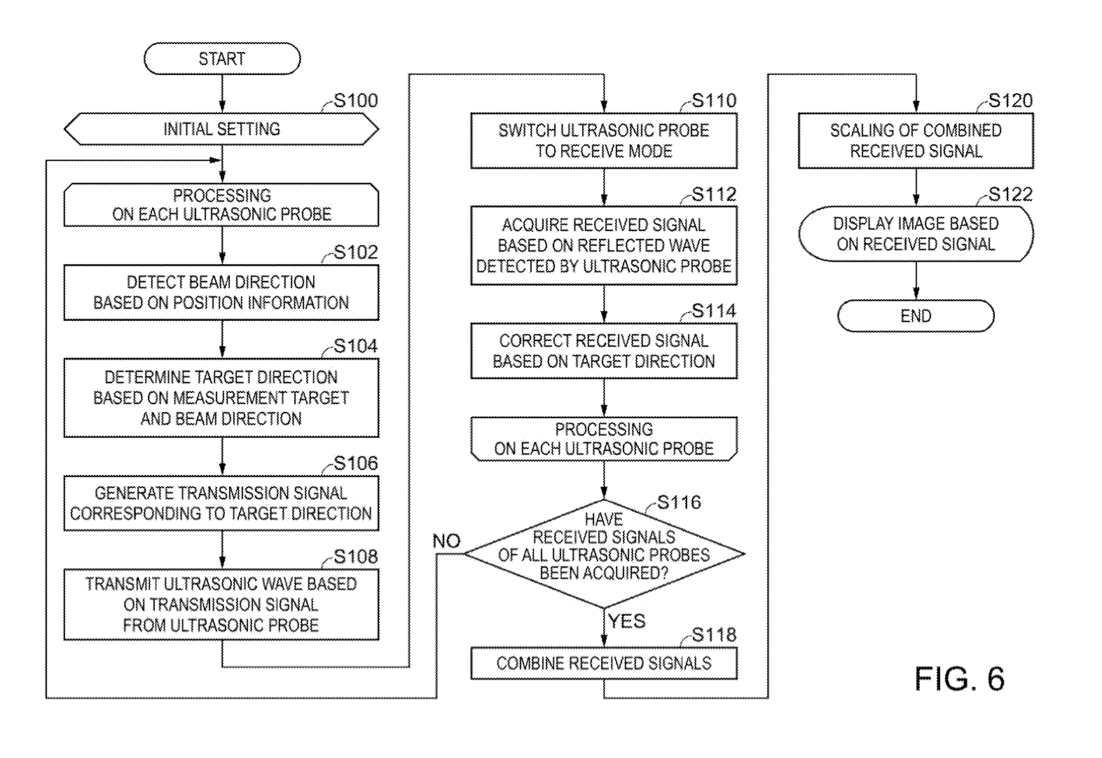

FIG. 6 is a flowchart showing the flow of the measurement process of the ultrasonic measurement apparatus 10. When the process starts, the CPU of the measurement control unit 50 performs initial setting for starting the measurement (step S100).

Then, the CPU causes each ultrasonic probe 20 to start the process, and detects a beam direction based on the position information of the ultrasonic probe 20 of interest (step S102) <detection step>.

Then, the CPU determines the target direction of the ultrasonic wave based on the measurement target and the beam direction (step S104).

Then, the CPU generates a transmission signal corresponding to the target direction (step S106) <transmission processing step>, and transmits an ultrasonic wave based on the transmission signal from the ultrasonic probe 20 of interest (step S108) <transmission step>.

Then, the CPU switches the ultrasonic probe 20 of interest to the receive mode (step S110).

Then, the CPU acquires a received signal based on the reflected wave detected by the ultrasonic probe 20 of interest (step S112) <acquisition step>.

Then, the CPU corrects the received signal based on the target direction of the ultrasonic wave (step S114) <correction step>.

Then, the CPU determines whether or not received signals of all ultrasonic probes 20 of interest have been acquired (step S116).

When the CPU determines that the received signals of all ultrasonic probes 20 of interest have not been acquired (No in step S116), the process returns to step S102 with the ultrasonic probe 20 whose received signal has not been acquired as a next target.

On the other hand, when it is determined that the received signals of all ultrasonic probes 20 of interest have been acquired (Yes in step S116), the CPU combines the acquired received signals of the ultrasonic probes 20 (step S118).

Then, the CPU performs various kinds of processing including scaling processing on the combined received signal (step S120)<combination step>.

Then, the CPU displays an image based on the received signal (step S122), and ends a series of measurement processes.

According to the embodiment described above, the following effects can be obtained.

(1) When the measurement of the measurement unit 15 for diagnosing the inside of the body using an ultrasonic wave is started by mounting the belt 18 on the body, reference beam directions of the plurality of ultrasonic probes 20 that are different depending on the state of contact with the body are detected, and ultrasonic waves toward a desired measurement target are transmitted to the respective ultrasonic probes 20 based on the reference beam directions. Therefore, an effort of the examiner to adjust a plurality of ultrasonic probes 20 so as to match the measurement target or an effort to hold the measurement unit 15, which is required in the related art, can be eliminated, and the degree of freedom to select the measurement point can be improved. As a result, since it is possible to shorten the time required for the measurement, it is possible to reduce the burden on the examiner.

(2) A reflected wave obtained by reflection of the transmitted ultrasonic wave within the body is received as a received signal in each ultrasonic probe 20 and is subjected to delay correction processing based on the information of the reference beam direction. Then, the plurality of received signals are combined and are displayed as an image. Accordingly, since the plurality of received signals are corrected and combined based on the information of each reference beam direction, it is possible to shorten the time required for the processing of the received signals.

While the embodiment of the invention has been described with reference to the diagrams, the specific configuration is not limited to the above-described embodiment, and various changes may be made in design without departing from the spirit of the invention. For example, measurement data formatted in a predetermined format may be stored in a storage device or may be transmitted to an information processing apparatus, such as a personal computer, without being limited to generating an image signal, such as a tomographic image, based on the combined received signal.

In addition, depending on the examination, one ultrasonic probe 20 or a plurality of ultrasonic probes 20 combined may be attached to the body using an adherence seal or the like, without being limited to providing the ultrasonic probe 20 on the belt 18.

In addition, the apparatus to perform the method described above may be realized by a single apparatus or may be realized by a plurality of apparatuses, and various aspects are included.

Each configuration and the combination of configurations in each embodiment are examples, and additions, omissions, replacement, and other changes to the configuration can be made without departing from the spirit and scope of the invention. In addition, the invention is not limited to the embodiment, but is limited only by the range of the appended claims.

The entire disclosure of Japanese Patent Application No. 2013-203606, filed Sep. 30, 2013 is expressly incorporated by reference herein.

What is claimed is:

1. An ultrasonic measurement apparatus, comprising:
an ultrasonic probe that transmits an ultrasonic wave and outputs a received signal based on a reflected wave of the ultrasonic wave;
a fixture that fixes the ultrasonic probe to a subject;
a position detecting unit that detects position information indicating a position of the ultrasonic probe with respect to the subject;
a transmission direction determining unit that determines a transmission direction of the ultrasonic wave based on the position information; and
a transmission signal generating unit that generates a transmission signal for transmitting the ultrasonic wave in the determined transmission direction.

2. The ultrasonic measurement apparatus according to claim 1, further comprising:
a received signal correcting unit that corrects the received signal based on the position information; and
a signal combining unit that combines the received signal corrected for each ultrasonic probe.

3. The ultrasonic measurement apparatus according to claim 1,
wherein the transmission signal generating unit generates the transmission signal including performing delay processing.

4. The ultrasonic measurement apparatus according to claim 2,
wherein the received signal correcting unit performs delay correction processing on the received signal of the ultrasonic probe based on the position information.

5. The ultrasonic measurement apparatus according to claim 1,
wherein the position detecting unit detects an installation angle of the ultrasonic probe with respect to the subject, and calculates the position information based on the detected installation angle.

6. The ultrasonic measurement apparatus according to claim 1,
wherein a plurality of the ultrasonic probes are provided, and
the position detecting unit detects a difference between installation angles of the ultrasonic probes adjacent to each other, and calculates the position information based on the detected difference.

7. The ultrasonic measurement apparatus according to claim 2, further comprising:
a display processing unit that generates an image based on the received signal combined by the signal combining unit and displays the generated image.

8. A measurement method, comprising:
detecting position information indicating a position of an ultrasonic probe with respect to a subject;
determining a transmission direction of the ultrasonic probe based on the position information;
generating a transmission signal for transmitting an ultrasonic wave in the determined transmission direction; and
transmitting the ultrasonic wave based on the transmission signal.

9. The measurement method according to claim 8, further comprising:
acquiring a received signal based on a reflected wave of the transmitted ultrasonic wave;
correcting the received signal based on the position information; and
combining the corrected received signal.
